(19) United States
(12) Patent Application Publication
(10) Pub. No.: US 2005/0249672 A1
(43) Pub. Date: Nov. 10, 2005

(54) Temporary tattoo for testing the sensitivity of skin to chemicals

(76) Inventor: John Anthony Bolbot, Bedfordshire (GB)

Correspondence Address: STITES & HARBISON PLLC, 1199 NORTH FAIRFAX STREET, SUITE 900, ALEXANDRIA, VA, 22314, US

(21) Appl. No.: 10/516,132

(22) PCT Filed: Jun. 11, 2003
(86) PCT No.: PCT/GB03/02514

§ 371 Date: Jun. 27, 2005

(30) Foreign Application Priority Data
Jun. 12, 2002 (GB) 0213437.7

(51) Int. Cl. A61K 49/00

(52) U.S. Cl. 424/9.81

(57) ABSTRACT

Potentially irritant or allergenic test materials are incorporated into predetermined areas (12) of a temporary tattoo (30) which is applied to skin (31). After a few days the tattoo (30) is removed and the exposed skin is examined. Reddened areas (32,36) can be correlated with test materials that were present in overlying areas of the tattoo, e.g. by comparison with a map (40) and key.

### TECHNICAL FIELD

The present invention relates to methods and means for testing the sensitivity of (generally human) skin to chemicals that may be present in cosmetics, particularly but not exclusively hair care products such as hair colouring products.

Mammalian skin may display a variety of reactions to materials by way of skin contact; when adverse, these reactions are usually described as irritation or sensitivity. They may or may not be mediated by the immune system.

Compounds which are used for the care or cosmetic embellishment of hair and to which some people are sensitive include phenylenediamine, resorcinol and nitrogen-containing compounds including ammonia salts; other examples would include the many proprietary cocktails of compounds mixed by individual hairdressers or supplied by commercial manufacturers for use on the hair.

### BACKGROUND ART

At present, the users of hair care products, particularly colourants, are usually advised to perform a preliminary skin sensitivity test by applying a sample to a small area of skin which is then inspected for signs of inflammation some time later (usually 24 or 48 hours). If there is no inflammation, the user may then proceed to treat his or her hair. After application of the test sample to the skin, it may be recommended that it be protected by an adhesive dressing in order to prevent its inadvertent removal (perhaps by scratching, rubbing or washing). Alternatively, such an adhesive dressing may be provided for the user, already impregnated with the skin test sample (see GB-A-2 325 521).

Such advice is commonly ignored. A prospective user of a hair colourant does not want to go round for a couple of days with an unsightly patch of coloured skin, or an unlovely plaster. Furthermore, if the advice is taken and skin sensitivity is revealed, this is not very informative. It just indicates that the subject is sensitive to something in the complex mixture of chemicals making up the cosmetic product.

### DISCLOSURE OF THE INVENTION

Broadly, the invention is based on the idea of incorporating material(s) to be tested into a temporary tattoo, which can then be worn on the skin for a period of time sufficient to reveal any physiological skin reaction to the material(s) in question. The novelty lies in the use of a temporary tattoo as the vehicle for such testing.

Conventional temporary tattoos can be quite durable, being water-resistant and usually lasting for several days. They can be quickly removed with cosmetic oils or alcohol.

A temporary tattoo is a picture, design or other artwork, possibly including colours, letters and numbers, which is applied to the skin and thereby effects an adornment which is not permanent in the traditional sense of a tattoo. There are three principle types of temporary tattoo which differ in the way they are applied to the skin. These are as follows:
1) Those which are hand-painted or drawn directly on the skin.
2) Those which are printed (or hand-painted or drawn) onto a substrate and then transferred to the skin some time later.
3) Those which are printed (or hand-painted or drawn) onto a transparent substrate which is subsequently stuck onto the skin.

For a type 1 temporary tattoo, the artwork is applied to the skin with brushes and/or pens or similar implements, using inks or dyes, in a fashion analagous to making an illustration on paper. A stencil can be used.

For a type 2 temporary tattoo, the artwork is usually printed in ink or dye onto a substrate (such as paper) having properties which allow the artwork to become dissociated from the substrate, some time after the printing, and transferred to skin (hence the alternative names decalcomania, decal or transfer). This transference is most commonly achieved by placing the printed substrate on the skin with the substrate uppermost and then wetting the substrate and applying a gentle pressure before peeling off the substrate, leaving the artwork adhered to the skin. Clearly, the substrate, inks and dyes must be selected such that they have properties which are appropriate to the transference procedure. Alternatively, there may be a transparent, water-resistant layer which covers the surface of the substrate and on top of which the artwork is printed. In this case, the transparent layer is transferred to the skin along with the artwork and thereafter forms a protective cover for the artwork, being uppermost on the skin when the substrate is peeled away. Alternatively, a transparent protective layer may be applied to the tattoo after transfer of the artwork to the skin by covering the artwork with a strip of transparent material or else by applying the transparent layer in the form of a solution from which the layer is deposited on the tattoo after evaporation of the solvent. A transparent protective layer may also be applied to a type 1 temporary tattoo by these methods.

For a type 3 temporary tattoo, the artwork is usually printed in ink or dye onto a transparent and water-resistant substrate which has an adhesive area that exceeds, overlaps or surrounds the artwork and assists, or is responsible for, its adherence to the skin (hence the alternative name, Stickers). A type 3 temporary tattoo is stuck onto the skin such that the artwork is in contact with the skin and the transparent substrate provides a protective adhesive layer.

A temporary tattoo (of any type) which is to be used for skin sensitivity testing may be produced so that certain designated areas of the artwork contain different test compounds (or test materials). These may be incorporated in the inks or dyes which form the portions of the design in these areas. Different areas could also contain the same test compound or material but at different concentrations. The person carrying out the test would keep a careful record or “tattoo map” detailing the precise distribution of the test materials in the tattoo. After the time allotted for the test, the tattoo may be removed with an appropriate reagent and the skin underneath examined for any signs of reaction. Comparing signs of sensitivity (e.g. skin reddening) with the map may enable the responsible compound(s) to be identified. Part of the tattoo may be less readily removed than the majority of the tattoo, so that the majority can be removed while leaving behind a minor amount (e.g. defining an outline) to assist in correlating any affected areas of skin with areas of the tattoo.

Clearly, the use of temporary tattoos for skin sensitivity testing need not be limited in application to the cosmetic treatment of hair. The invention can also be applied to other types of skin sensitivity testing, particularly allergy-testing. In this case the designated areas would serve in an analogous fashion to that described above except in being vehicles for a different kind of test compound or material.

Temporary tattoos based on water slide transfer technology have been around since the 1980s. Almost any printing technique (litho, letterpress, screen process etc.) can be employed to create an image. Sheet fed litho is the preferred method because of the very thin ink film that can be achieved. Water slide paper, typically of around 170 gsm, is commercially available. It is calendared on one side and coated with a water soluble layer on the printing side. After printing the tattoo image, the image area is coated with a solvent based lacquer; screen processing is the preferred method for this application. The effect of this is to fuse all the printed image into a film. At this stage, once dry this is a water slide transfer. The lacquer film containing the printed image can be released by immersing the transfer in water. The transfer can then be applied to almost any rigid non-permeable surface, adhesion being achieved by the exclusion of air. Using special inks this technique is widely used in the ceramic industry to decorate china, porcelain etc.

In order to use such a transfer as a temporary tattoo some form of adhesive is generally employed to enable the transfer to adhere to human skin. A water-based acrylic dispersion adhesive, screen printed through a very fine mesh, to achieve the thinnest film thickness possible, is the preferred method. Some form of clear protective film is desirable to prevent dust etc. contamination.

Compounds to be tested for skin sensitivity may be incorporated in the inks used for printing the image. Alternatively they may be present in one or more additional layers.

### BRIEF DESCRIPTION OF THE DRAWINGS

FIG. 1 is a schematic view for explaining the production process for an embodiment of the invention.

FIG. 2 is a schematic view of a temporary tattoo embodying the invention, applied to a person's upper arm.

FIG. 3 is a view corresponding to FIG. 2, showing the upper arm after removal of the temporary tattoo.

FIG. 4 shows a “map” for use with the tattoo shown in FIG. 2.

### BEST MODE FOR CARRYING OUT THE INVENTION

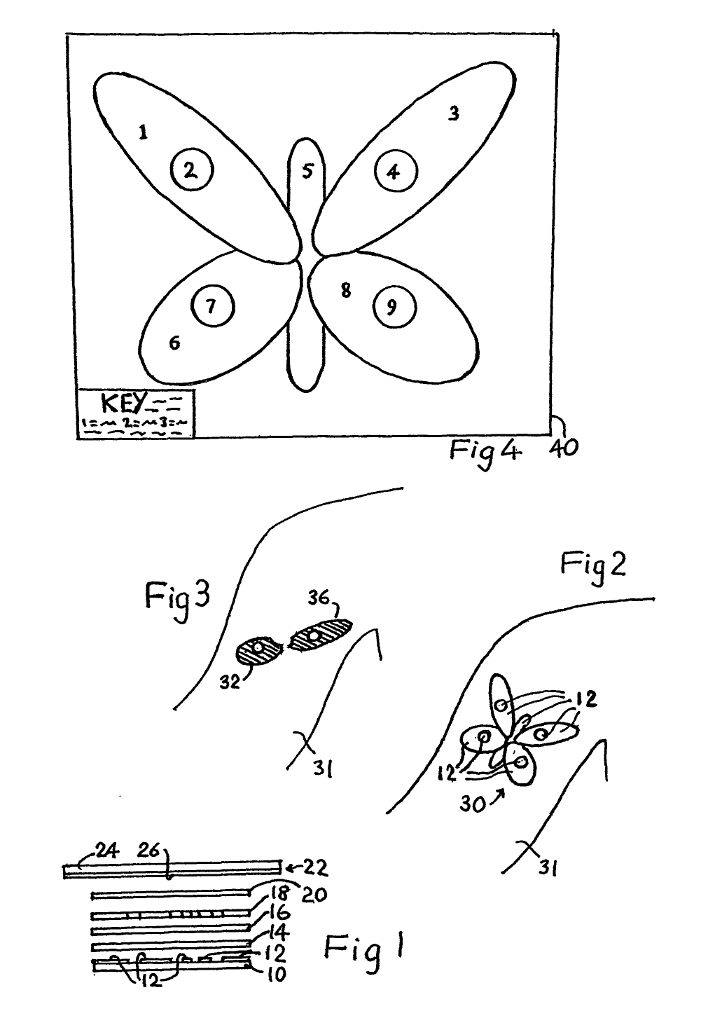

FIG. 1 shows the various layers of material involved in the production of an embodiment of a temporary tattoo as used in the present invention. A base substrate 10 is a commercially-available water slide paper, of 176 gm⁻². On its printing side, it has been printed with an image, using a plurality of coloured inks. Ink-bearing portions are indicated by 12. The printing used conventional four-colour process lithography, using inks specified for cosmetic use.

Next, a layer of lacquer 14 was applied to fuse the coloured image, and form a waterproof layer. It was applied by screen printing a solvent-based lacquer composition.

Next, a HEC cellulose barrier layer 16 was formed by screen printing. On top of this, a test layer 18 was applied, also by screen printing. This is a layer of innocuous medium, with regions in register with the printed areas 12 which contain one or more test compounds. The innocuous medium is suitably a polyvinyl pyrrolidone material. The barrier layer 16 is to prevent materials from this layer 18 from bleeding through to the base paper 10.

A layer of medical grade acrylic water-based adhesive 20 was screen printed over the test layer. (The material of the adhesive was chosen so that it would form a pervious layer, enabling transmission of the test compounds.) Finally, a release paper 22 was applied. This is a high density polythene clear film laminate protective layer 24, coated with a layer of silicone 26 on the side which contacts the adhesive layer 20.

For use, the release layer is peeled away, and the adhesive layer is pressed against the skin surface to receive the tattoo. Water is applied to the exposed face of the base substrate paper 10 until it can be peeled away, leaving the printed image 30 defined by the printed areas 12.

FIG. 2 shows an example of such a temporary tattoo 30 which has been applied to the skin 31 of an upper arm. In this case, it represents a butterfly. FIG. 4 is a “map” of the tattoo image, with the different areas numbered. Underlying a numbered region, there is a corresponding region of the test layer 18, which may include a test compound. The map of FIG. 4 shows nine different areas so, in principle, the tattoo could be testing nine different compounds and/or concentrations. However, it may be preferable for there to be a smaller number of compounds, each present in more than one area, to provide a degree of reassurance. In this example, it is assumed that the areas numbered 3 and 6 in FIG. 4 bear the same compound, and that the user is sensitive to this compound.

After a suitable period (e.g. two days), the tattoo is removed, and the exposed skin 31 is examined. FIG. 3 shows the presence of a pair of reddened areas 32,36. Comparison with the map 40 shown in FIG. 4 can provide information about the test compounds involved. The map will generally be provided with a key, for relating the different areas to the test compounds. This key may be present on the map, or it could be kept by a manufacturer of the product.

1. A kit comprising (a) a cosmetic product containing a plurality of chemicals; and (b) means for applying a temporary tattoo having a plurality of regions containing different ones and/or different mixtures and/or different concentrations of said plurality of chemicals so that the tattoo when on a user's skin delivers said chemicals to corresponding underlying regions of the skin.

2. A kit according to claim 1 further including a map of the tattoo with indicia indicating which of said, chemicals are in which regions thereof.

3. A skin sensitivity testing kit comprising (a) means for applying to skin a temporary tattoo with a predetermined design comprising a plurality of predetermined regions containing different test materials and/or different concentrations of test materials so that the tattoo when on a user's skin delivers said test material(s) to corresponding underlying regions of the skin; and (b) a map of the tattoo with indicia indicating the predetermined regions.

4. A kit according to claim 3 wherein said means for applying a temporary tattoo is a substrate bearing a transfer-type tattoo.

5. A method of skin sensitivity testing comprising (a) applying to skin a temporary tattoo with a predetermined design comprising a plurality of predetermined regions containing different test materials and/or different concentrations of test materials so that the tattoo delivers said test material(s) to corresponding underlying regions of the skin; (b) observing the skin, optionally after removal of the tattoo, to determine whether any of said underlying regions exhibits a sensitivity reaction.

6. A method according to claim 5 wherein there is a map of the tattoo with indicia indicating the predetermined regions, and step (b) includes a step of comparing the skin to which the tattoo was applied with the map to correlate any observed sensitivity reaction with the corresponding predetermined region of the tattoo and hence with the test material and/or test material concentration of that predetermined region.

7. A method according to claim 5 wherein said temporary tattoo is applied using a substrate bearing a transfer-type tattoo which is transferred to the skin.
